(19) United States
(12) Patent Application Publication
(10) Pub. No.: US 2009/0181363 A1
(43) Pub. Date: Jul. 16, 2009

(54) NON-INVASIVE DETECTION OF FISH VIRUSES BY REAL-TIME PCR

(76) Inventor: Arun K. Dhar, Sykesville, MD (US)

Correspondence Address: MOORE & VAN ALLEN PLLC, P.O. BOX 13706, Research Triangle Park, NC, 27709, US

(21) Appl. No.: 12/158,096

(22) PCT Filed: Dec. 21, 2006
(86) PCT No.: PCT/US2006/048976

§ 371 Date: Dec. 31, 2008

Related U.S. Application Data
(60) Provisional application No. 60/752,930, filed on Dec. 21, 2005.

(51) Int. Cl. C12Q 1/70; C12Q 1/68

(52) U.S. Cl. 435/5; 435/6

(57) ABSTRACT

A real-time assay coupled with a non-invasive tissue sampling was developed for the detection and quantification of fish viruses. As a proof of principles, data were presented for the detection and quantification of infectious hypodermal necrosis virus (IHNV) in trout. The primers were designed for IHNV nucleocapsid (N), and surface glycoprotein (G) genes, and trout &bgr;-actin and elongation factor-l&agr; (EF-I &agr;) were used as internal control for the assay. The reaction conditions for the real-time RT-PCR were optimized using cDNA derived from IHNV-infected Epithelioma papulosum cyprinid (EPC) cells. Using both N- and G-gene primers, IHNV was successfully detected in liver, kidney, spleen, adipose tissue and pectoral fin samples of laboratory-challenged and wild samples. The dissociation curves with a single melting peak at expected temperature (85° C. for the N-gene and 86.5° C. for the G-gene) confirmed the specificity of the N- and G-gene amplicons. The IHNV N- and the G-gene expression levels in different tissues of laboratory challenged samples were in the order of spleen, liver, kidney, adipose tissue and pectoral fin, however in the field-collected samples the order of gene expression was liver, kidney, pectoral fin, adipose tissue, and spleen. The N- and G-gene expressions in spleen were found to be dramatically lower in the field-collected samples compared to the laboratory-challenged samples indicating a potential difference in the IHNV replication in the laboratory as opposed to field conditions. The real-time PCR assay was found to be rapid, highly sensitive, and reproducible. Based upon the ability to detect the virus in pectoral fins a non-invasive detection method for IHNV and other fish viruses is developed. Such a non-invasive tissue sampling coupled with real-time PCR assay is very valuable for large-scale virus screening of fish in aquaculture facilities as well as for epidemiological studies.

### BACKGROUND OF THE DISCLOSURE

Fisheries' contribution to global food supply has become increasingly important as world population increases (FAO 2000) and consumers recognize the importance of the omega-3 fatty acids supplied by fish. The supply of seafood from capture fisheries is declining globally and there is an urgent need to enhance aquacultural productivity worldwide. Management of aquatic animal health is a pre-requisite for sustainable and increased development of global aquaculture. Diseases of animals in commercial fisheries are caused by diverse biotic and abiotic factors. Among them, diseases caused by viruses are of particular importance. Disease outbreaks often pose major challenges for sustainable development in aquaculture. A case in point is salmonid aquaculture. In 2002, the US salmon industry was virtually destroyed by an outbreak of infectious salmon anemia virus.

Viral diseases are major obstacles to salmon farming. For example, diseases caused by infectious hematopoietic necrosis virus (IFfNV), infectious pancreatic necrosis virus (IPNV), infectious salmon anemia virus (ISAV), viral hemorrhagic septicemia virus (VHSV) and nodaviruses caused severe economic losses in salmonid aquaculture. Large scale and rapid monitoring offish in commercial fisheries is useful in reducing the chances of entry of these viral pathogens in the production system. Due to the extensive losses caused by these viruses in salmon and trout aquaculture facilities, several methods have been developed for detecting the IHNV, IPNV, ISAV and VHSV (Winton 1991). These include isolating the virus from candidate fish in established cell lines and confirming the identity by serum neutralization, enzyme-linked immunosorbent assay (ELISA), in situ hybridization using biotinylated probes, immunohistochemical and immunogold labeling, and RT-PCR (OIE 2000). Among these methods, RT-PCR is the most sensitive and rapid method of detection. However, quantification of the target gene by conventional RT-PCR is laborious, time consuming and relies on post-PCR analysis of the amplified product.

Recently, fluorescence-based real-time PCR has been developed for the detection and quantification of viruses (Bustin 2000; Mackay et aL 2002). Real-time PCR has greater sensitivity than conventional PCR, requires very little initial RNA, and thus, becomes very useful when dealing with limited amounts of tissue samples. In addition, it has a wide dynamic range of detection, does not require post-PCR analysis, and has high throughput ability (Bustin 2000). Real-time PCR detection can be applied to large-scale screening of viruses in commercial aquaculture.

Different methods are employed to detect the amplicons generated by real-time RT-PCR. These include detection using DNA-binding fluorochromes, such as SYBR Green I (INVITROGEN), linear oligoprobes, 5′ nuclease oligoprobes, molecular becons, and self-fluorescing amplicons (Mackay et al. 2002). Among them, detection by direct fluorochromes, such as SYBR® Green I, is the simplest, since it does not require the design of fluorogenic oligoprobes, and is the least expensive method. Higher melting temperatures of the expected amplicons allows discrimination of target amplicons from primer dimer in SYBR Green realtime PCR (Ririe et al. 1997). Real-time PCR is valuable for the detection of viral pathogens in plants and animals including humans (reviewed in (Mackay et al. 2002; Niesters 2002). However, the potential of real-time PCR in detecting viruses in different fish species is only beginning to be realized. For example, real-time PCR using TaqMan™ probes (Applied Biosystems) have been developed for the detection and quantification of IHNV in trout (Overturf et al. 2001), and real-time RT-PCR using SYBR Green chemistry has been developed for ISAV in Atlantic salmon and rainbow trout (Munir and Kibenge 2004). These studies generally involved invasive tissue sampling, including tissue biopsies from brain, kidney, heart, spleen, liver, gills, and pyloric caeca. Among the non-invasive tissue sampling sites, mucus was examined for IHNV presence in rainbow trout that were experimentally infected with IHNV by waterborne exposure or injection (LaPatra et al. 1989). Using RT-PCR analysis, detected IHNV titers reached a maximum level between 24 to 84 hours post-infection and then gradually declined.

The disclosure describes the use of non-invasive tissues, such as the pectoral fin clip, for the detection of IHNV and other viral pathogens by real-time PCR assay. The combination of non-invasive tissue sampling and real-time PCR can be used in multiple applications, including (1) large-scale screening of broodstock fish for viral pathogens in commercial hatcheries, (2) screening fish for virus resistance and susceptibility in breeding programs, (3) in epidemiological studies to monitor the prevalence and potential outbreak of viral diseases in commercial fisheries and wild fish populations, and (4) to examine the expression of fish gene(s) using fin clip.

Viral diseases are a major problem for both wild and aquacultured salmonids. Thus these diseases impact the environment and the fish farming communities. Among the major viral pathogens of salmonids, IHNV is one of the most important viruses. Biological, serological, and nucleic acid-based detection methods have been developed for the detection of IHNV in salmonids. All these methods require invasive tissue sampling. In fact, lethal sampling is routinely used where the animals are sacrificed to determine if the pathogen is present. Among the existing methods of IHNV detection, conventional reverse transcriptase polymerase chain reaction (RT-PCR) assay is the most sensitive. However, detection and quantification of IHNV by conventional RT-PCR is laborious and relies on post-PCR analysis of the amplified product(s).

### BRIEF SUMMARY OF THE SEVERAL VIEWS OF THE DRAWINGS

FIG. 1 is an image of an agarose gel separation of IHNV N- and G-gene amplicons amplified using real-time RT-PCR. The primers for the N-gene amplification were N737F and N843R and for the G-gene amplification were G1035 and G1 147R (Table 1). M=100 bp DNA ladder, I=cDNA derived from IHNV-infected EPC cells, and C=control cells. The arrow indicates the N- and the G-gene amplicons.

FIG. 2 is a series of amplification profiles and dissociation curves of the IHNV G-gene (FIG. 2A)₃ the IHNV N-gene (FIG. 2B), and trout β-actin gene (FIG. 2C) amplified from infectious hematopoietic necrosis virus (IHNV)-infected trout tissue samples. The melting temperatures (Tₘ) are indicated alongside the dissociation curves of the corresponding amplicons. K=Kidney, L=Liver, S=Spleen, A=Adipose tissue and P=pectoral fin.

FIG. 3 is a pair of bar graphs showing relative viral load of infectious hematopoietic necrosis virus (IHNV)-infected laboratory challenged and field collected trout tissue samples. K=Kidney, L=Liver, S=Spleen, A=Adipose tissue, and P=pectoral fins. Note: In real-time RT-PCR, ΔCₜ is inversely related to viral load. Therefore, lower the value of ΔQ, higher the IHNV load in the tissue. FIG. 3A shows resultes for the IHNV-G gene, and FIG. 3B shows results for the IHNV-N gene.

### DETAILED DESCRIPTION

Methods are described herein that overcome the limitations of conventional RT-PCR. These methods relate to the potential of non-invasive tissue sampling coupled with real-time RT-PCR to improve viral detection in fish. Since real-time RT-PCR and conventional RT-PCR are the most sensitive among current methods available for virus detection and require very small tissue samples, they can be used for timely and large-scale screening for viruses in salmonids and other fish. In addition, real-time RT-PCR can be automated and has high throughput capability making it even more appealing for large-scale salmonid farming and epidemiological studies. The development of the detection kits for viral and pathogen detection in animals using noninvasive tissue sampling is an important aspect of the methods described herein.

By way of example is salmonid aquaculture, where viral diseases are major obstacles to successful salmon and trout farming. Diseases caused by infectious hematopoietic necrosis virus (IHNV), infectious pancreatic necrosis virus (IPNV), infectious salmon anemia virus (ISAV), and viral hemorrhagic septicemia virus (VHSV) have caused severe economic losses in salmonid aquaculture (LaPatra et al. 2001; Cipriano and Miller 2003). Large-scale and economical screening of fish in commercial fisheries is desirable for reducing the chances of entry and subsequent spread of these viral pathogens in commercial production systems. Additionally, the method allows for routine monitoring throughout the course of culture to set disease status and empirically determine threshold values for the disease and production system. Due to extensive losses caused by these viruses in salmon and trout aquaculture facilities, several methods have been developed for detecting IHNV, IPNV, ISAV and VHSV as an aid to disease control (Winton 1991; Bootland and Leong 1999). These methods include culturing the virus from candidate fish using established cell lines with the identity confirmed by a serum neutralization assay, enzyme-linked immunosorbent assay (ELISA), in situ hybridization using a biotinylated probe, immunohistochemical or immunogold labeling, or RT-PCR (Bootland and Leong 1999; OIE 2003). Among these methods, RT-PCR is the most sensitive and rapid method of detection. However, detection and quantification of the target gene by conventional RT-PCR is laborious, time consuming, and relies on post-PCR analysis of an amplified product.

Recently, fluorescence-based real-time PCR has been utilized for the detection and quantification of viruses (Bustin 2000; Mackay et al. 2002). Real-time PCR has a large dynamic range, high sensitivity, needs no post-PCR amplification processing, has greater sensitivity than conventional PCR, and is amenable to high throughput. In addition, it requires only a small quantity of RNA, and thus, becomes very useful when dealing with limited amounts of tissue (Bustin 2000; Wong and Medrano 2005). Thus, real-time PCR detection can be applied to large-scale screening of viruses in commercial aquaculture. A disadvantage to real-time PCR is that it requires expensive instruments and high cost reagents. In addition, due to its high sensitivity, proper experimental design and laboratory hygiene are imperative for successful results. However, since its first introduction, the price of a real-time thermocycler has fallen nearly 50% (−$50,000 in 1998-1999 to −$25,000 in 2005). The cost of reagents can also be reduced by proper optimization {e.g., reducing the reaction volume from 50 to 25 μL, (Dhar et al. 2001; Dhar et al. 2002)); and making in-house reaction mixtures (Karsai et al. 2002). In addition, the time and labor saved through high-throughput screening can, over time, offset the initial cost. Significantly, due to extreme sensitivity, the pathogen can be detected at an earlier time to enable better prevention or management of epizootics.

Several of the examples provided have involved Infectious Hematopoietic Necrosis Virus (IHNV), but the methods described herein can apply to any systemic virus and other viruses that shed viral proteins to the circulatory system of the animal. IHNV is the type species in the genus Novirhabdovirus within the family Rhabdoviridae and infects several species of wild and cultured salmonids. IHNV causes severe epidemics in young fish, infects adults, or remains asymptomatic in carriers. IHNV is endemic throughout the Pacific Northwest from Alaska to California and into Idaho. The virus has spread to Asia and Europe through the movement of infected fish and contaminated eggs (Winton 1991). The IHNV genome contains a negative-sense, single-stranded RNA of ˜11 Kb which contains six genes in the following 3′ to 5′ order: nucleocapsid (N), polymerase-associated phosphoprotein (P), matrix (M), surface glycoprotein (G), non-virion protein (NV), and virus RNA dependent RNA polymerase (L) (Morzunov et al. 1995; Schuetze et al. 1995). SYBR Green real-time RT-PCR uses primers targeting the N, G and the L genes. The N-gene is the first expressed and most abundant protein present during IHNV infection (Bootland and Leong 1999). Therefore, the N-gene is a good target for early detection of IHNV. Compared to the N-gene, the G-gene is expressed later in IHNV infection (9-10 hours post-infection). The middle of the G-gene (also called mid-G) was found to be variable among different IHNV isolates making this section of the G-gene potentially a good marker for phylogenetic analysis of IHNV isolates (Troyer et al. 2000; Kurath et al. 2003; Garver et al. 2003). Therefore, primers flanking mid-G are useful to differentiate IHNV isolates based on the difference in the melting temperature (Tₘ) of the amplicons in SYBR Green realtime RT-PCR. Melting temperatures of the amplicons have recently been used to differentiate feline calicivirus isolates, a single stranded RNA virus (Helps et al. 2002), and bluetongue virus of sheep, a dsRNA containing virus (Orru et al. 2004). The polymerase (L) gene of IHNV is an early gene and conserved among the IHNV isolates. Therefore, the L-gene can serve as a good candidate for early detection of IHNV. In addition, the fact that all the sequences of L-gene available in the database are almost identical means that primers based on the L-gene is able to amplifying all isolates of IHNV. On the other hand, primers designed flanking the variable region of IHNV G-gene can be used to differentiate different strains of IHNVV based on the difference in the melting temperature (Tₘ). A routine screening offish in a farm or in the wild by real-time PCR using primers that can differentiate strains can predict the emergence of a new strain based on the difference in the Tₘ values of the amplicons.

Thus the methods described herein are useful in screening fish in large-scale commercial operations as well as for epidemiological and field studies. Another embodiment of the method is a non-invasive, highly sensitive, rapid diagnostic kit for IHNV as well as a method to differentiate IHNV strains. Such a technology has a general applicability to other fish viruses, bacteria, and pathogens.

The disclosure relates, in one embodiment, to a method for detection of a pathogen, wherein an optimized PCR primer is used to detect nucleic acid by applying real-time PCR to samples of non-invasive tissues of an animal.

The disclosure relates, in another embodiment, to a method for detection of a fish pathogen wherein an optimized PCR primer is used to detect nucleic acid by real-time PCR in non-invasive tissues of an animal such as blood, mucus, feces, skin, or fin clip. The disclosure relates, in yet another embodiment, to a real-time PCR method for detection of a viral disease wherein the sample is taken from a non-invasive tissue such as fin clip, blood sample, mucus scrape, feces or skin sample.

The disclosure relates, in still another embodiment, to a real-time PCR method for differentiation of viral strains wherein the sample is taken from a non-invasive tissue such as fin clip, blood sample, mucus scrape, feces or skin sample.

The disclosure relates, in another embodiment, to a kit for detection of a pathogen based on samples recovered from a non-invasive tissue sampling based on real-time PCR detection methods.

The disclosure relates, in another embodiment, to a kit for detection of fish pathogens based on samples recovered from a non-invasive tissue sampling based on real-time PCR detection methods.

The disclosure relates, in another embodiment, to a kit for detection of fish viral pathogens based on samples recovered by non-invasive tissue sampling based on real-time PCR detection methods.

The disclosure relates, in another embodiment, to a kit for differentiation of fish viral pathogens based on samples recovered from a non-invasive tissue sampling based on real-time PCR detection methods.

The disclosure relates, in another embodiment, to a kit for detection offish bacterial pathogens based on samples recovered from a non-invasive tissue sampling based on real-time PCR detection methods.

### EXAMPLES

The following examples are provided for illustration only and not by way of limitation.

### Example 1

Primer Optimization for Real-Time RT-PCR

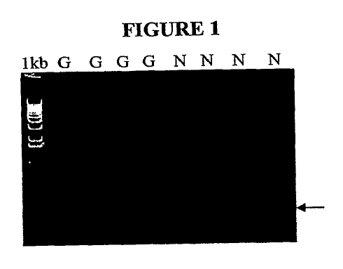

Primers for IHNV N- and G-genes were designed based on the sequence of IHNV reference strain WRAC, GenBank accession number L40883 (Table 1). Total RNA was isolated from IHNV-infected EPC (Epithelioma papillosum cyprinid) cell culture, and cDNA synthesized using MultiScribe reverse transcriptase (PE Applied Biosystems). Two sets of N- and G-gene primers (Table 1) were screened using three combinations (50, 300 and 900 nM) of the forward and reverse primers. The N- and G-gene primer sequences chosen were conserved across different isolates of IHNV. The N-gene primer combination (N737F and N843R) and the G-gene primer combination (G1035 and G1 147R) provided the lowest cycle threshold (Cₜ) values and the optimal primer concentration was 300 nM of each both forward and reverse primers. The melting curves for both the N- and G-gene amplicons showed a single peak at their expected melting temperatures. Neither the N- nor G-gene primers provided any amplification with cDNA derived from control EPC cells. The amplified cDNAs for the N- and G-genes showed 107 and 113 bp bands in an agarose gel, respectively (FIG. 1). The N- and G-gene amplicons were gel purified and then sequenced. The nucleotide sequences of both genes showed 100% similarity with the IHNV GenBank entry, accession number L40883, on which the primers were designed. This indicated that the N- and G-gene amplicons amplified by real-time RT-PCR was indeed of viral origin.

TABLE 1

List of primers used for real-time RT-PCR assay for the
detection and quantification of infectious
hematopoietic necrosis virus (IHNV).

| | Primer | Primer Sequence (5′-3′) | % | | Amplicon |
|---|---|---|---|---|---|
| Gene | Name | [SE ID NO] | GC | TM | Size (bp) |
| IHNV | N316F | ACCTTCGCAGATCCCAACAAC [1] | 52 | 64 | 126 |
| N-gene | N441R | TGTGGCCATCTTGTCCACATC [2] | 52 | 64 | |
| | N737F | TGTGCATGAAGTCAGTGGTGG [3] | 52 | 63 | 107 |
| | N843R | CCTGCTCATCATGACACCGTA [4] | 52 | 62 | |
| IHNV | G296F | TCCACAAAGTCCTGTACCGCA [5] | 52 | 64 | 114 |
| G-gene | G409R | TGTCATACGCCCCTGCTTCTT [6] | 52 | 64 | |
| | G1035F | CGCTATGCACAAAGGCTCCAT [7] | 52 | 65 | 113 |
| | G1147R | ATTTCGTGAAGCTGGTAGCGC [8] | 52 | 64 | |
| Trout β-actin, | 1301F | CCCAAACCCAGCTTCTCAGTCT [9] | 55 | 64 | 113 |
| AF157514 | 1413R | TGCTTCACCGTTCCAGTTGTG [10] | 52 | 64 | |
| Trout EF-Iα | 136F | TGATCTACAAGTGCGGAGGCA [11] | 52 | 64 | 101 |
| factor 1-α, | 236R | CAGCACCCAGGCATACTTGAA [12] | 52 | 63 | |
| AF498320 | | | | | |

At 50 mM Na⁺

### Example 2

Detection of IHNV in different tissue samples from laboratory-challenged and naturally infected trout samples

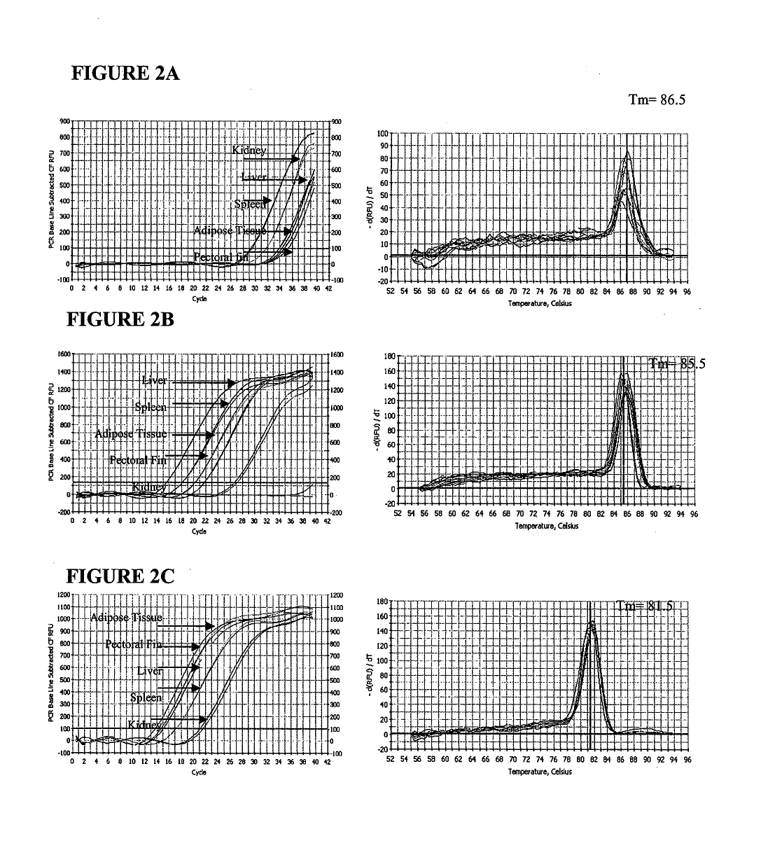

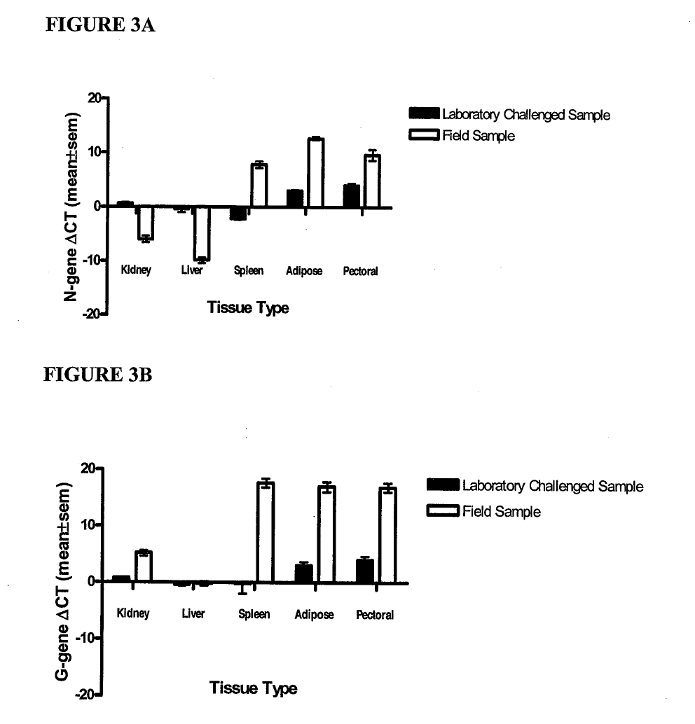

The IHNV N- and G-genes were detected in kidney, liver, spleen, adipose tissue, and pectoral fins of both laboratory-challenged and naturally infected trout. The amplification profiles and the dissociation curves of N-and G-gene amplicons in all five different tissues are shown in FIG. 2. The melting curves of both N- and G-gene amplicons showed a single peak at 85.5° C. and 86.5° C., respectively, indicating the specificity of the PCR products. The relative expression of the N- and G-genes in different tissues of laboratory- challenged and naturally infected trout sample is shown in FIG. 3. In general, liver, kidney, and spleen tissues had a higher level of expression (therefore lower ΔCₜ value) compared to adipose tissues and pectoral fins for both N- and the G-genes in laboratory-challenged and naturally infected trout. However, there were noticeable differences among different tissues in the same trout as well as between the laboratory-challenged as opposed to naturally infected samples. For example, in the laboratory-challenged samples the N-gene expression in kidney and liver was almost equivalent. But the spleen had a 2^{3.07}-fold higher, adipose tissues had a 2^{7.83}-fold lower, and pectoral fins had 2^{3.72}-fold lower expression compared to kidney tissues (FIG. 3A). On the other hand, in the naturally infected samples, liver tissues had the highest level of expression followed in decreasing order by kidney, spleen, pectoral fin, and adipose tissue. The expression of the N-gene in the later three tissues was dramatically lower (2¹⁵ to 2¹⁹-fold) compared to kidney tissue. It was also notable that the highest level of virus in laboratory-challenged trout was found with spleen but wild trout has highest level in the liver and kidney. The G-gene expression did not show a noticeable difference between the kidney, liver, and spleen in the laboratory-challenged samples. However, the adipose tissue and the pectoral fin had a 2^{2.7} to 2^{3.7}-fold lower level of expression compared to kidney tissue. In the naturally infected samples, the G-gene expression was dramatically lower in the spleen, pectoral fin and the adipose tissues compared to kidney tissues.

The difference in the expression of N-gene among different tissues of laboratory-challenged and naturally infected samples can be due to route of entry of the virus (injection in the laboratory-challenged as opposed to natural route of entry for the filed samples), dose of infection (2×10⁷ pfu/mL in the laboratory-challenged samples vs. unknown, and presumably lower, dose of infection in the wild) as well as the variation in the progress of the disease in wild as opposed to controlled laboratory conditions. Nevertheless, these variations did not undermine the fact that SYBR Green real-time RT-PCR can detect IHNV in the pectoral fin clip samples of trout. Therefore, fin clip sampling served as a non-invasive method for early detection of IHNV. These data showed the use of a non-invasive sampling technique in detecting IHNV for large-scale screening of fish in commercial fisheries for control and management of disease in aquaculture. Additionally, these methods can be used for epidemiological and field studies to aid the control of the disease in nature.

### Example 3

Optimization of real-time RT-PCR conditions using primers based on structural (glycoprotein, G and nucleocapsid, N) and non-structural (RNA-dependent-RNA polymerase, L) genes of IHNV.

The initial optimizations of the real-time RT-PCR conditions were performed using total RNA derived from IHNV-infected EPC (Epithelioma papulosum cyprinid) cell line. EPC cells were inoculated with IHNV using a virus inoculum at 2.5×10⁷ pfu/mL (IHNV Strain 220.90) and following a published protocol (LaPatra et al. 1994). Virus inoculated and control cell cultures were maintained at 17° C. in minimum essential medium supplemented with 2% fetal bovine serum. Four days post-inoculation, control and virus-inoculated cells were harvested and 500 μL TRI Reagent™ (Molecular Research Center, Inc., Ohio) were added before storing the cells at −80° C.

Total RNA was isolated from control and IHNV-infected EPC cells following the TRI Reagent RNA isolation protocol. The RNA pellets were dissolved in TE buffer and the yield measured by using a spectrophotometer (Biorad). The RNA quality were assessed in a 1% formaldehyde agarose gel following a standard protocol (Sambrook et al. 1989). The cDNA syntheses were carried out in a 40 μL reaction volume containing 1 μg total RNA, IX RT-PCR buffer, 1 mM dNTPs (PE Applied Biosystems), 0.75 μM oligo dT, 4 U of RNase inhibitor (PE Applied Biosystems) and 5 U of MultiScribe reverse transcriptase (PE Applied Biosystems). The cDNA reaction mixture was diluted to 1:10 dilutions using DNase, RNase free molecular biology grade water and 1 μL of the diluted cDNA was taken for each amplification reaction. Two sets of primers for amplifying the IHNV N- and G-genes using SYBR Green real-time RT-PCR were tested by real-time RT-PCR (see results below). These primers were designed based on a published sequence of the virus flanking a conserved region in the respective gene (GenBank accession number L40883, IHNV reference strain WRAC) using Primer Express Software version 1.0 (PE Applied Biosystem). One additional set of primers were designed and evaluated for the N- and G-genes. The primers for the G-gene were designed based on the variable domain (mid G) of this gene. Three sets of primers were designed for the L-gene of IHNV using the sequence from the same accession number (L40883, IHNV reference strain WRAC).

The SYBR Green real-time RT-PCR amplifications were carried out in a Bio-Rad MyiQ™ device (Bio-Rad Laboratories, Inc., Richmond, Calif.). Three different primer concentrations were evaluated in the real-time RT-PCR assay using a checkerboard (all possible combinations of 50, 300, and 900 nM concentrations of forward and reverse primers). After primer optimization, one set of primers was selected for each of N-, G- and L-genes for subsequent work. The reaction mixture contained 12.5 μL of 2X SYBR Green Supermix (iQ™ SYBR Green Supermix), optimal concentrations of forward and reverse primers and 1 μL of the 1:10 diluted cDNA in a 25 μL reaction volume. These amplifications were carried out in a 96 well microplate with three replicates per sample. The thermal profile for SYBR Green real-time RT-PCR was 95° C. for 3 min followed by 40 cycles of 95° C. for 10 sec and 60° C. for 1 min.

### Example 4

Detect and quantify IHNV in different tissues of laboratory-challenged rainbow trout collected by non-invasive (fin clip) vs. invasive sampling (liver, kidney, spleen and adipose tissue) at 3 days post-challenge.

Viral challenge was performed by injecting specific pathogen-free rainbow trout (Oncorhynchus mykiss Walbaum) intraperitoneally with approximately 10⁷ pfu/mL of IHNV (IHNV Strain 220-90, LaPatra et al 1994). Animals were sacrificed at 7 days post-challenge. Pectoral fin, mucus, and gill samples were collected before dissecting the animals to collect liver, kidney, adipose tissue, and spleen tissues. All tissue samples (50-100 mg) were collected in TRI reagent and stored at −80° C. until RNA isolation was performed. Tissue samples from control (sham injected) fish were collected in a similar manner. An aliquot of samples from all the above tissues were collected to determine the IHNV load by plaque assay (see below). There were 4 fish for sham injection and 6 fish for the IHNV-injection treatment. Thus, a total of 50 samples (4 sham injected fish, 6 IHNV-injected fish, 5 different tissue types per fish) were collected.

RNA isolation, cDNA synthesis, and the optimized real-time PCR conditions, as described above, were applied to the detection and quantification of IHNV in trout challenged in the laboratory. One optimal primer set for each of the N-, G-, and L-genes were used for the detection and quantification of IHNV. In addition to these three viral genes, one internal control gene, such as trout β-actin, were tested along side the viral gene in each 96-well plate. There were two to three replicates for each reaction.

After a SYBR Green PCR run, data acquisition and subsequent data analyses were done using the MyiQ Real-Time PCR Detection System (Software Version 1.0). In the iCycler® (Bio-Rad), the fluorescence of SYBR Green against the internal passive reference dye (ΔRₙ) was measured at the end of each cycle. A sample was considered positive when ΔRₙ exceeded the threshold value. The threshold value was set at the midpoint of ΔRₙ vs. cycle number plot. The threshold cycle (Cₜ) was defined as the cycle at which a statistically significant increase in Rn was first detected.

In order to determine the relative viral load in different tissues of IHNV -infected samples of trout, the Q values of N-, G-, and L-genes were subtracted from the geometric mean Ct values of β-actin gene of the corresponding tissues. The differences in the Cₜ value of the viral genes and the corresponding internal control were expressed as ΔCₜ. The ΔCₜ normalizes to correct for any difference in the amount of total RNA added to the cDNA reaction and the efficiency of the reverse transcription reaction. The difference in the ΔCₜ for one tissue type (e.g., kidney) compared to the ΔCₜ of another tissue (e.g., spleen) were expressed as a ΔΔQ. 2^{ΔΔCT} value allowed to measuring the relative IHNV load in one tissue type over the other. A ΔΔCt value difference of 3.3 was considered to be equivalent to a 10-fold difference in IHNV load. The Cₜ values were exported into an Excel spread sheet and analyzed by the ANOVA test using SPSS version 11.5.

### Example 5

Comparing the IHNV load in different tissues collected by non-invasive (fin clip) and invasive (liver, kidney, spleen and adipose tissue) sampling from laboratory-challenged trout at 3 days post-challenge.

The viral load (relative and absolute copy number) was measured in different tissues of trout to determine the general applicability of this non-invasive approach for IHNV detection. These samples were taken at 3 days post-challenge as described in Example 4.

### Example 6

Comparing the IHNV load in different tissues collected by non-invasive (fin clip, mucus, blood, feces) and invasive (gill, liver, kidney, spleen) sampling from laboratory- challenged trout at different time points post-challenge.

The viral load (relative and absolute copy number) is measured in different tissues of trout to determine the general applicability of this non-invasive approach for IHNV detection. These samples are taken at the time points as described above. Comparing the viral load in different tissues at different time point enables one to determine when the earliest time point the virus can be detected in fin clip, mucus or blood compared to gill, liver, kidney, spleen and adipose tissues. This information indicates the suitability of using samples collected in a noninvasive manner as opposed to invasive sampling for large scale screening. In addition, the relationship of viral loads in the various tissues aids in both the description of the viral pathogenesis in the fish and the suitability of the non-invasive test being developed.

### Example 7

Validating the assay by comparing the IHNV load determined by real-time RT-PCR to the viral load determined by plaque assay in fin clip, liver, kidney, feces, adipose tissue, and spleen of laboratory challenged trout.

The plaque assay is performed using homogenates from different tissues (fin clip, mucus, liver, kidney, spleen, adipose tissue and feces) of IHNV-challenged trout at different time points post-challenge following published protocol (LaPatra et al. 1994). A parallel section of these tissues is used to determining the IHNV load using real-time RT-PCR.

The IHNV load is measured by the standard plaque assay and compared to the IHNV load determined by the real-time RT-PCR assay. Since viral load determined by the real-time PCR assay cannot provide information on the infectivity of the virus, plaque assay is conducted to measure the level of infectious virus in these tissues. This method provides earlier detection than that achieved using the plaque assay and can be adapted into a kit format for eventual commercialization.

### Example 8

Kit for Non-Invasive Detection of IHNV in Trout or Salmon.

A kit is provided to the person taking the sample, either in the field, aquaria, or in the lab. The kit provides a step-by step protocols on how to collect samples in a non-invasive manner for real-time PCR so a person, regardless of technical background, can obtain and preserve a sample in a form that can be transported to the site of analysis without significant degradation.

In a first embodiment, a fin clip sample is collected from the pectoral fin of a fish using a sterile forceps or a punch hole. The forceps or the punch hole is sterilized using 70% ethanol in between sample collections. The fin clip sample is preserved in an Eppendorf tube containing RNA isolation buffer and kept frozen at —SO ⁰C until further use.

In a second embodiment, blood (100-500 μï) is drawn using a sterile 1 ml tuberculin syringe with a 25 gauge needle. Separate syringe and needle are used for each fish. Immediately after collection, blood samples are mixed with RNA isolation buffer in an Eppendorf tube and kept frozen at −80° C. until further use by the laboratory technician.

In a third embodiment, mucus samples are collected from the fish using a Q-tip with a sterile cotton swab. Separate Q-tips are used to collect samples from each fish. After collection, the cotton swab is put into RNA isolation buffer (e.g. TRI Reagent, MRC, Inc., Ohio) in an Eppendorf tube and kept frozen at −80° C. until further use by the laboratory technician.

In a fourth embodiment, fecal samples are collected from the fish, and put into RNA isolation buffer (e.g. TRI Reagent, MRC, Inc., Ohio) in an Eppendorf tube and kept frozen at −80° C. until further use by the laboratory technician.

In a fifth embodiment, skin samples are collected from the fish, and put into RNA isolation buffer (e.g. TRI Reagent, MRC, Inc., Ohio) in an Eppendorf tube and kept frozen at −80° C. until further use by the laboratory technician.

An analysis kit provides the laboratory technician to quickly set up and run real-time PCR on any of a number of real-time PCR instruments. For an RNA virus, total RNA is isolated from any collected samples and cDNA is synthesized for real-time RT-PCR. For a DNA virus, non-invasive samples collected in a way similar to RNA virus, as described above, except that samples are preserved in DNA isolation buffer (e.g. DNAZoI, MRC, Inc., Ohio). Total genomic DNA is isolated from the sample before performing the real-time PCR. The real-time PCR kit contains an optimized PCR mixture, forward and reverse primers, and positive control to ensure that the reaction mixture works and a detailed protocol on how to set up the reaction in a realtime thermocycler.

### Example 9

ISAV Detection

The ISAV of salmon is an RNA virus containing eight segments of negative-strand RNA. In order to detect ISAV, primers are designed for the polymerase gene, encoded by the segment 2, and or nucleoprotein gene, encoded by the segment 3, and or non-structural protein gene, encoded by the segment 8 to detect ISAV by real-time RT-PCR. Based on the highest sensitivity, appropriate primers are used in the ISAV detection kit.

### Example 10

Differentiation of Viral Strains Offish by Real-Time PCR

In order to identify different strains of a virus, primers are designed based on genes that show hypervariation. For example, in the IHNV G-gene, there are domains that are highly variable and flanked by conserved regions. Primers are designed based on the conserved regions and flanking the variable domains. Real-time PCR amplicons derived from such a variable domain shows a difference in the melting temperature (Tₘ). Since the Tₘ values are unique for any nucleic acid, different strains have different Tₘ values which are used as a signature for the identification of that particular strain.

### Example 11

Detection of Bacterial Pathogens

Bacterial diseases offish are one of the limitations in successful fish farming. Bacterial diseases such as streptococcal infection caused by Streptococcus iniae often causes mass mortalities in tilapia and striped bass. A tentative diagnosis of streptococcal infection can be made from the history and clinical signs. However, for confirming the diagnosis, the animals are sacrificed to collect brain, spleen, kidney, or liver tissues for bacterial culture. These procedures involve invasive techniques, are time consuming and are less sensitive. A rapid and highly sensitive detection method for Streptococcus is developed based on real-time PCR and non-invasive tissue sampling (fin clip, blood samples, feces, mucus).

References

The following technical articles are referred to herein.

Bootland L M, Leong J C (1999) Infectious haematopoietic necrosis virus. In: Woo P, Bruno D (eds) Fish Diseases and Disorders. CAB International, pp 57-121.

Bustin S (2000) Absolute quantification of mRNA using real-time reverse transcription polymerase chain reaction assays. J Mol Endrocrinol 25:169-193.

Cipriano R, Miller O J (2003) Infectious salmon anemia: The current state of our knowledge. In. USDA Technical Bulletin, pp 1-11.

Dhar A K, Roux M M₅ Klimpel K R (2001) Detection and quantification of infectious hypodermal and hematopoietic necrosis virus and white spot virus in shrimp using real-time quantitative PCR and SYBR Green chemistry. J Clin Microbiol 39:2835-2845.

Dhar A K, Roux M M₅ Klimpel K R (2002) Quantitative assay for measuring the Taura syndrome virus and yellow head virus load in shrimp by real-time RT-PCR using SYBR Green chemistry. J Virol Methods 104:69-82.

FAO (2000) The State of World Fisheries and aquaculture (SOFIA). In. FAO Fisheries Department.

Garver K A, Troyer R M3 Kurath G ( 2003) Two distinct phylogenetic clades of infectious haematopoietic necrosis virus overlap within the Columbia River basin. Dis Aquat Organ 55:187-203.

Helps C, Lait P, Tasker S, Harbour D (2002) Melting curve analysis of feline calicivirus isolates detected by real-time reverse transcription PCR. J Virol Methods 106:241-244.

Karsai A, Muller S, Platz S, Hauser M T (2002) Evaluation of homemade SYBR Green I reaction mixture for real-time PCR quantification of gene expression. Biotechniques 32:790-796.

Kurath G, Graver K A, Troyer R M, Emmenegger E J, Einer-Jensen K, Anderson E D (2003) Phylogeography of infectious haematopoietic necrosis virus in North America. J Gen Virol 84:803-814.

LaPatra S, Batts W, Jones G, Shewmaker W, Winton J (2001) Absence of risk associated with the movement of processed rainbow trout from an area where infectious haematopoietic necrosis virus is endemic. In: OIE Conference: Risk analysis in aquatic animal health. OIE, pp 240-245.

LaPatra S E, Lauda K A, Jones G (1994) Antigenic variants of infectious haematopoietic necrosis virus and implications for vaccine development. Dis Aquatic Org 20:119-126.

LaPatra S E, Rohovec J S, Fryer J L (1989) Detection of infectious hypodermal necrosis virus in fish mucus. Fish Pathology 14:197-202.

Mackay I M, Arden K E, Nitsche A (2002) Real-time PCR in virology. Nucl Acids Res 30:1292-1305.

Morzunov S P, Winton J R, Nichol S (1995) The complete genome structure and phylogenetic relationship of infectious haematopoietic necrosis virus. Virus Res 38:175-192.

Munir K, Kibenge F (2004) Detection of infectious salmon anaemia virus by realtime RT-PCR. J Virol 117:37-47.

Niesters H G M (2002) Clinical virology in real-time. J Clin Virol 25:S3-S12.

OJJE (2000) Diagnostic Manual for Aquatic Animal Diseases. OIE, Paris.

OIE (2003) Chapter 2.1.2 Infectious haematopoietic necrosis. Manual of diagnostic tests for aquatic animals 2003.

Orru G, Santis P D, Solinas F, Savini G, Piras V, Caporale V (2004) Differentiation of Italian field and South African vaccine strains of bluetongue virus serotype 2 using real-time PCR. J Virol Methods 122:37-43.

Overturf K, LaPatra S, Powell M (2001) Real-time PCR for the detection and quantitative analysis of IHNV in salmonids. J. Fish Dis 24:325-333.

Ririe K M₅ Rasmussen R P, Wittwer C T (1997) Product differentiation by analysis of DNA melting curves during the polymerase chain reaction. Anal Biochem 270:154-160.

Sambrook J₃ Fritsch E₃ Maniatis T (1989) Molecular Cloning: A laboratory manual, 2 edn. Cold Spring Harbor Press, Cold Spring Harbor.

Schuetze H, Enzmann P J, Kuchling R, Mundt E₃ Niemann H, Mettenleiter T (1995) Complete genomic sequence of the fish rhabdovirus infectious haematopoietic necrosis virus. J Gen Virol 14:2519-2527.

Troyer R M, LaPatra S E₃ Kurath G (2000) Genetic analysis reveals unusually high diversity of infectious haematopoietic necrosis virus in rainbow trout aquaculture. J Gen Virol 81:2823-2832.

Winton J R (1991) Recent advances in detection and control of infectious hematopoietic necrosis virus in aquaculture. Ann Rev Fish Dis:83-93.

Wong M L, Medrano J F (2005) Real-time PCR for mRNA quantitation. Biotechniques 39:75-85.

The disclosure of every patent, patent application, and publication cited herein is hereby incorporated herein by reference in its entirety.

While this subject matter has been disclosed with reference to specific embodiments, it is apparent that other embodiments and variations can be devised by others skilled in the art without departing from the true spirit and scope of the subject matter described herein. The appended claims include all such embodiments and equivalent variations.

What is claimed is:

1. A method for detection of a pathogen in an organism, such method comprising analysis of non-invasive tissues of the organism by real-time PCR or real-time RT-PCR.

2. A method according to claim 1, wherein the pathogen is a virus.

3. A method according to claim 1, wherein the virus is a fish virus.

4. A method according to claim 2, wherein the virus is an RNA virus.

5. A method according to claim 4, wherein the RNA virus is selected from infectious hypodermal necrosis virus, infectious pancreatic necrosis virus, infectious salmon anemia virus, viral hemorrhagic septicemia virus.

6. A method according to claim 4, wherein the RNA virus is identified based on unique melting temperature of dsRNA formed from a probe oligo and the targeted regions of the virus.

7. A method according to claim 2, wherein the virus is a DNA virus.

8. A method according to claim 7, wherein the DNA virus is selected form channel catfish virus, Koi herpes virus, lymphocystis virus.

9. A method according to claim 7, wherein the DNA virus is identified based on the unique melting temperature of the amplicon or the unique sequence of the targeted region of the virus to which the probe anneals.

10. A method according to claim 1, wherein the pathogen is a bacterium.

11. A method according to claim 1, wherein the organism is a fish.

12. A method according to claim 11, wherein the fish is selected from salmon, rainbow trout, koi, carp, catfish, bass, sea bass, tilapia, flounder, halibut, sturgeon.

13. A method according to claim 10, wherein the bacterium is chosen from Streptococcus iniae, pathogenic Aeromonas spp. (e.g., A. salmonicid\acute{α}), and Pseudomonas spp. (e.g., P. fluoresceins, P. putida, P. anguilliseptica, P. chlororaphis, Flexibacter spp., Flavobacterium spp., Vibrio spp).

14. A method according to claim 1, wherein the tissue sampled is selected from blood, skin, mucus, feces or fin clips.

15. A kit for detection of a pathogen, such kit comprising analysis of non-invasive tissue of an organism by real-time PCR or real-time RT-PCR.

16. A kit according to claim 15, wherein the pathogen is a virus.

17. A kit according to claim 16, wherein the virus is a fish virus.

18. A kit according to claim 16, wherein the virus is an RNA virus.

19. A kit according to claim 18, wherein the RNA virus is chosen from infectious hypodermal necrosis virus, infectious pancreatic necrosis virus, infectious salmon anemia virus, viral hemorrhagic septicemia virus.

20. A kit according to claim 16, wherein the virus is a DNA virus.

21. A kit according to claim 20, wherein the DNA virus is selected form channel catfish virus, koi herpes virus, lymphocystis virus.

22. A kit according to claim 15, wherein the pathogen is a bacterium.

23. A kit according to claim 15, wherein the organism is a fish.

24. A kit according to claim 23, wherein the fish is selected from salmon, rainbow trout, koi, carp, catfish, bass, sea bass, tilapia, flounder, halibut, sturgeon.

25. A kit according to claim 22, wherein the bacterium is chosen from Streptococcus iniae, pathogenic Aeromonas spp. (e.g., A. salmonicida), and Pseudomonas spp. (e.g., P.fluorescens, P. putida, P. anguilHseptica, P. chlororaphis).

26. A kit according to claim 15, wherein the non-invasive tissue is selected from blood, skin, mucus, feces or fin clips.

27. A method for differentiation of viral strains, such method comprising analysis of noninvasive tissues of an organism by real-time PCR or real-time RT-PCR.


SEQUENCE LISTING (12 sequences)

SEQ ID NO: 1 - DNA, 21 bp, Artificial Sequence
  Synthetic construct
accttcgcagatcccaacaac

SEQ ID NO: 2 - DNA, 21 bp, Artificial Sequence
  Synthetic Construct
tgtggccatcttgtccacatc

SEQ ID NO: 3 - DNA, 21 bp, Artificial Sequence
  Synthetic Construct
tgtgcatgaagtcagtggtgg

SEQ ID NO: 4 - DNA, 21 bp, Artificial Sequence
  Synthetic Construct
cctgctcatcatgacaccgta

SEQ ID NO: 5 - DNA, 21 bp, Artificial Sequence
  Synthetic Construct
tccacaaagtcctgtaccgca

SEQ ID NO: 6 - DNA, 21 bp, Artificial Sequence
  Synthetic Construct
tgtcatacgcccctgcttctt

SEQ ID NO: 7 - DNA, 21 bp, Artificial Sequence
  Synthetic Construct
cgctatgcacaaaggctccat

SEQ ID NO: 8 - DNA, 21 bp, Artificial Sequence
  Synthetic Construct
atttcgtgaagctggtagcgc

SEQ ID NO: 9 - DNA, 22 bp, Artificial Sequence
  Synthetic Construct
cccaaacccagcttctcagtct

SEQ ID NO: 10 - DNA, 21 bp, Artificial Sequence
  Synthetic Construct
tgcttcaccgttccagttgtg

SEQ ID NO: 11 - DNA, 21 bp, Artificial Sequence
  Synthetic Construct
tgatctacaagtgcggaggca

SEQ ID NO: 12 - DNA, 21 bp, Artificial Sequence
  Synthetic Construct
cagcacccaggcatacttgaa